(19) United States
(12) Patent Application Publication
(10) Pub. No.: US 2025/0255484 A1
(43) Pub. Date: Aug. 14, 2025

(54) MATERIALS AND METHODS FOR LUMINESCENCE-BASED CARBON DIOXIDE SENSING

(71) Applicant: The General Hospital Corporation, Boston, MA (US)

(72) Inventors: Xiaolei Li, Boston, MA (US); Juan Pedro Cascales Sandoval, Somerville, MA (US); Emmanouil Rousakis, Charlestown, MA (US); Conor L. Evans, Charlestown, MA (US)

(21) Appl. No.: 18/856,679

(22) PCT Filed: Apr. 17, 2023
(86) PCT No.: PCT/US2023/065836

§ 371 Date: Oct. 14, 2024

Related U.S. Application Data
(60) Provisional application No. 63/331,829, filed on Apr. 16, 2022.

(51) Int. Cl. A61B 5/00; A61B 5/083

(52) U.S. Cl. CPC A61B 5/0071; A61B 5/0062; A61B 5/0082; A61B 5/0836; A61B 5/6803; A61B 5/6846; A61B 5/742; A61B 2560/0223; A61B 2560/0462

(57) ABSTRACT

A device for carbon dioxide monitoring is disclosed. The device comprises: a photoluminescent carbon dioxide-sensitive probe comprising a polymer matrix and a sensing dye; a photon source configured to direct photons at the probe; a photodetector configured to detect light emitted from the probe when the photon source directs photons at the probe; a carbon dioxide permeable light redirection layer, wherein the carbon dioxide-sensitive probe is positioned between the light redirection layer and the photodetector; and a controller in electrical communication with the photon source and the photodetector, the controller being configured to execute a program stored in the controller to calculate a level of carbon dioxide adjacent the probe from an electrical signal received from the photodetector. In one form, the polymer matrix comprises a polymer selected from the group consisting of acrylate polymers, methacrylate polymers, polyurethane polymers, and blends and copolymers thereof.

### CROSS-REFERENCE TO RELATED APPLICATIONS

This application is based on, claims benefit of, and claims priority to U.S. Application No. 63/331,829 filed on Apr. 16, 2022, which is hereby incorporated by reference herein in its entirety for all purposes.

### STATEMENT REGARDING FEDERALLY SPONSORED RESEARCH

This invention was made with government support under one or more of grant number FA9550-17-1-0277, awarded by the Air Force Office of Scientific Research; and grant number HU0001-17-2-0009, awarded by the Henry M. Jackson Foundation for the Advancement of Military Medicine, Inc. The government has certain rights in the invention.

### BACKGROUND OF THE INVENTION

### 1. Field of the Invention

This invention relates to devices, materials, and methods for analyte monitoring, and more particularly to devices, materials, and methods for carbon dioxide monitoring.

### 2. Description of the Related Art

Carbon dioxide (CO₂) plays various roles in the human body such as respiratory drive, regulation of blood pH, and affinity of hemoglobin for oxygen. Monitoring CO₂ partial pressure (pCO₂) in breath (capnography) and blood is of immense importance for both medical diagnosis and treatment of human diseases such as respiratory and metabolic disorders. For example, the adequacy of spontaneous and mechanical ventilation is usually evaluated by measuring the CO₂ concentration in arterial blood. However, the current “gold standard” method to obtain this reading relies on the invasive process of arterial blood gas sampling through the placement of arterial lines. A non-invasive alternative to arterial gas sampling is to monitor pCO₂ on the skin surface or exhaled breath, which can reduce or altogether eliminate the need for blood gas sampling, decreasing the risk for patient co-morbidities and improving patient comfort. However, current technology based on infrared sensors, requires large and expensive equipment, long bed-side calibration procedures, immobile patients, etc.

Accordingly, there is a need to provide miniaturized sensors that can be incorporated into wearable devices and that can find wide application, such as continuous capnography or transcutaneous CO₂ sensors. Optical CO₂ sensors based on luminescent materials can offer several advantages, such as accurate detection of CO₂ levels as well as enormous potential for miniaturization.

### SUMMARY OF THE INVENTION

The present invention meets the foregoing needs by providing devices, materials, and methods for analyte (e.g., carbon dioxide) monitoring.

In one aspect, the disclosure provides a device for carbon dioxide monitoring. The device comprises: a photoluminescent carbon dioxide-sensitive probe comprising a polymer matrix and a sensing dye; a photon source configured to direct photons at the probe; a photodetector configured to detect light emitted from the probe when the photon source directs photons at the probe; and a controller in electrical communication with the photon source and the photodetector, wherein the controller is configured to execute a program stored in the controller to calculate a level of carbon dioxide adjacent the probe from an electrical signal received from the photodetector, and wherein the polymer matrix comprises a polymer selected from the group consisting of acrylate polymers, methacrylate polymers, polyurethane polymers, and blends and copolymers thereof.

In one embodiment of the device, the polymer matrix comprises a hydrophobic polymer. In one embodiment of the device, the polymer matrix comprises a polymer selected from the group consisting of alkyl methacrylate polymers. In one embodiment of the device, the polymer matrix comprises poly (propyl methacrylate) (PPMA).

In another aspect, the disclosure provides a device for carbon dioxide monitoring. The device comprises: a photoluminescent carbon dioxide-sensitive probe comprising a polymer matrix and a sensing dye; a photon source configured to direct photons at the probe; a photodetector configured to detect light emitted from the probe when the photon source directs photons at the probe; a carbon dioxide permeable light redirection layer, wherein the carbon dioxide-sensitive probe is positioned between the redirection layer and the photodetector; and a controller in electrical communication with the photon source and the photodetector, the controller being configured to execute a program stored in the controller to calculate a level of carbon dioxide adjacent the probe from an electrical signal received from the photodetector.

In one embodiment of the device, the light redirection layer comprises a silicone film including a pigment. The pigment can be white. In one embodiment of the device, the light redirection layer scatters light. In one embodiment of the device, the light redirection layer reflects light. In one embodiment of the device, the light redirection layer scatters light and reflects light. In one embodiment, the device further comprises a heater for increasing a temperature of the light redirection layer and/or the probe. In one embodiment of the device, the photons from the photon source of higher wavelength than required for exciting the sensing dye are filtered using a short-pass filter. In one embodiment of the device, light emitted from the photon source is filtered by a long-pass filter covering the photodetector. In one embodiment, the device further comprises a partially or a completely air-impermeable layer positioned between the carbon dioxide-sensitive probe and the photodetector. In one embodiment, the device further comprises a partially or a completely air-impermeable outer layer. In one embodiment of the device, the polymer matrix comprises a polymer selected from the group consisting of acrylate polymers, methacrylate polymers, silicate polymers and sol-gels, polyurethane polymers (e.g., in the form of films or foams), and blends and copolymers thereof.

In yet another aspect, the disclosure provides a device for carbon dioxide monitoring. The device comprises: a photoluminescent carbon dioxide-sensitive probe comprising a polymer matrix and a sensing dye; a first photon source configured to direct photons at a first wavelength at the probe; a second photon source configured to direct photons at a second wavelength at the probe, wherein the second wavelength is different from the first wavelength; a photodetector configured to detect light emitted from the probe when the first photon source and the second photon source direct photons at the probe; and a controller in electrical communication with the first photon source and the second photon source and the photodetector, wherein the controller is configured to execute a program stored in the controller to calculate a level of carbon dioxide adjacent the probe from an electrical signal received from the photodetector.

In one embodiment of the device, an excitation spectrum of the sensing dye has an isosbestic point at the first wavelength. In one embodiment of the device, directing photons at the first wavelength at the probe provides a normalization factor to account for variations in brightness of the polymer matrix. In one embodiment of the device, the controller is configured to execute the program stored in the controller to calculate the level of carbon dioxide adjacent the probe using a fluorescence ratio providing a metric that is proportional to the level of carbon dioxide adjacent the probe and is normalized using the normalization factor. In one embodiment of the device, the controller is configured to execute the program stored in the controller to run a calibration algorithm such that the device can report the level of carbon dioxide adjacent the probe via wired or wireless communication. In one embodiment of the device, the calibration algorithm uses different calibration parameters depending on whether a derivative of the fluorescence ratio is positive or negative. In one embodiment of the device, the controller is configured to execute the program stored in the controller to calculate the level of carbon dioxide adjacent the probe by detecting intensity of the emission excited at the first wavelength and the second wavelength. In one embodiment of the device, the first photon source and the second photon source are modulated by a sinusoidal voltage and an intensity of a fluorescence response of the sensing dye is defined as an amplitude of a measured sinusoidal response, which is extracted via multiple linear regression.

In any embodiment of the devices for carbon dioxide monitoring, the photodetector detects a fluorescence response of the sensing dye that provides tissue pCO₂. The sensing dye can comprise a pH-sensitive fluorescent dye. In one embodiment, the pH-sensitive fluorescent dye comprises 8-hydroxy-1,3,6-pyrenetrisulfonic acid trisodium salt (HPTS). The sensing dye can comprise a pH-sensitive fluorescent dye in anion form and a quaternary ammonium cation. The carbon dioxide-sensitive probe can comprise a phase transfer reagent co-embedded with the sensing dye within the polymer matrix. The phase transfer reagent can have a volumetric ratio (v/v) with respect to the sensing dye of greater than 0% to 40%, or a volumetric ratio (v/v) with respect to the sensing dye of greater than 0% to 20%, or a volumetric ratio (v/v) with respect to the sensing dye of greater than 0% to 10%, or a volumetric ratio (v/v) with respect to the sensing dye of 2% to 8%.

In any embodiment of the devices for carbon dioxide monitoring, the phase transfer reagent provides water required for production of carbonic acid to protonate the pH-sensitive fluorescent dye. The phase transfer reagent can comprise an alkylammonium hydroxide. The phase transfer reagent can comprise tetraoctylammonium hydroxide. In any embodiment of the devices for carbon dioxide monitoring, the carbon dioxide-sensitive probe can have a storage stability such that no significant decrease of sensitivity when the carbon dioxide-sensitive probe is stored under ambient and dark conditions for at least seven days.

In any embodiment of the devices for carbon dioxide monitoring, the device can be an optical transcutaneous device. In any embodiment of the devices for carbon dioxide monitoring, the device can further comprise a display in electrical (wired or wireless) communication with the controller, wherein the controller is configured to execute the program stored in the controller to display the level of carbon dioxide on the display. The controller can be configured to execute the program stored in the controller to store values of the level of carbon dioxide at a plurality of times over a time period. In any embodiment of the devices for carbon dioxide monitoring, the device can further comprise a display in electrical communication with the controller, wherein the controller is configured to execute the program stored in the controller to display values of the level of carbon dioxide at a plurality of times over a time period.

In any embodiment of the devices for carbon dioxide monitoring, the device can be insertable in a body part of a patient. In any embodiment of the devices for carbon dioxide monitoring, each photon source, the photodetector, and the controller can be disposed in or on a support structure. In any embodiment of the devices for carbon dioxide monitoring, the device can include a band structured for securing the support structure to a body part of a patient. In any embodiment of the devices for carbon dioxide monitoring, the device includes a structure for securing the support structure into or on the nose or mouth. In any embodiment of the devices for carbon dioxide monitoring, the device includes a structure for securing the support structure into or on the group of a nasal tube, nasal cannula, mask, helmet, tracheal tube, or breathing apparatus.

In any embodiment of the devices for carbon dioxide monitoring, the device can further comprise a light guide for directing photons from each photon source at the probe. The light guide can be an optical fiber. In any embodiment of the devices for carbon dioxide monitoring, the device can further comprise a light guide for directing light emitted from the probe at the photodetector. The light guide can be an optical fiber.

Any embodiment of the devices for carbon dioxide monitoring, can be

included in a kit for monitoring carbon dioxide, wherein the kit comprises one or more additional photoluminescent carbon dioxide-sensitive probes. Each photoluminescent carbon dioxide-sensitive probe can be provided in a package that does not transmit photons of a wavelength that causes emission of light by the probe. The package can include an indicator of the useful lifetime of the photoluminescent carbon dioxide-sensitive probe. Any embodiment of the devices for carbon dioxide monitoring, can be included with a medical instrument, wherein the device is attached to the medical instrument.

In yet another aspect, the disclosure provides a method for end-tidal carbon dioxide monitoring. The device comprises: a photoluminescent carbon dioxide-sensitive probe comprising a polymer matrix and a sensing dye within a device or device extension that would be positioned within or close to the nostril or mouth, or incorporated into a cannula, mask, or helmet; a first photon source configured to direct photons at a first wavelength at the polymer matrix probe; a second photon source configured to direct photons at a second wavelength at the probe, wherein the second wavelength is different from the first wavelength; a photodetector configured to detect light emitted from the probe when the first photon source and the second photon source direct photons at the probe; and a controller in electrical communication with the first photon source and the second photon source and the photodetector, wherein the controller is configured to execute a program stored in the controller to calculate a level of carbon dioxide adjacent the probe from an electrical signal received from the photodetector.

In still another aspect, the disclosure provides a method for detecting a concentration of an analyte. The method comprises: (a) providing a device comprising: (i) a probe including a polymer matrix and a sensing dye, (ii) a first photon source configured to direct photons at a first wavelength at the probe, (iii) a second photon source configured to direct photons at a second wavelength at the probe, wherein the second wavelength is different from the first wavelength, and (iv) a photodetector configured to detect light emitted from the probe when the first photon source and the second photon source direct photons at the probe; and (b) calculating a concentration of the analyte adjacent the probe based on the light emitted from the probe detected by the photodetector and an isosbestic point from an excitation spectra of the sensing dye at the first wavelength within a physiological range of the analyte.

In any embodiment of the methods, step (b) can further comprise calculating the concentration of the analyte adjacent the probe based on a normalization factor determined by directing photons at the first wavelength at the probe to account for variations in brightness of the polymer matrix. In any embodiment of the methods, step (b) can further comprise calculating the concentration of the analyte adjacent the probe based on a luminescence ratio providing a metric that is proportional to the concentration of analyte adjacent the probe and is normalized using the normalization factor. In any embodiment of the methods, step (b) can further comprise calculating the concentration of the analyte adjacent the probe based on a calibration algorithm such that the device can report the concentration of analyte adjacent the probe via wired or wireless communication. The calibration algorithm can use different calibration parameters depending on whether a derivative of the luminescence ratio is positive or negative.

In any embodiment of the methods, step (b) can further comprise calculating the concentration of the analyte adjacent the probe by detecting the luminescence emission intensity excited at the first wavelength and the second wavelength. In any embodiment of the methods, step (b) can further comprise calculating the concentration of the analyte adjacent the probe by modulating by a sinusoidal voltage and an intensity of a luminescence response of the sensing dye is defined as an amplitude of a measured sinusoidal response, which is extracted via multiple linear regression. The analyte can be selected from the group consisting of molecular oxygen, carbon dioxide, nitric oxides, dissolved analytes in plasma and tissue, and hydrogen ions. The analyte can be selected from the group consisting of molecular oxygen and carbon dioxide and mixtures thereof. The analyte can be carbon dioxide. The concentration of the analyte can be calculated as pCO₂.

It is an advantage of the disclosure to provide bright, luminescent CO₂ sensing materials which are sensitive in the physiological CO₂ range (0-50 mmHg) wherein the materials are found to be photostable, such that their signal remains high during prolonged use.

It is another advantage of the disclosure to provide a noninvasive lightweight wired or wireless wearable device which, in conjunction with the sensing materials, can continuously monitor transcutaneous CO₂ partial pressure. The luminescence from the CO₂ sensing materials can be excited and detected via the device to reversibly detect changes in CO₂ within the physiological range. The sensing materials of the present disclosure also present important material properties useful in wearable CO₂ sensing applications. For example, the sensing materials are mechanically robust so the material can be formulated into different form factors. Additionally, for skin-worn devices, the material can be insensitive to changes in humidity to allow for consistent performance across different body locations and physiological and environmental variations.

Non-limiting examples of use of the technology of the present disclosure include: measurement of transcutaneous tissue CO₂ and oxygen partial pressure on burn, diabetic or post-surgical recovery patients, capnography, end-tidal CO₂ monitoring, continuous arterial gas sensor, etc.

These and other features, aspects and advantages of various embodiments of the present disclosure will become better understood with regard to the following description, appended claims, and accompanying Figures.

### BRIEF DESCRIPTION OF THE DRAWINGS

FIG. 1A shows an embodiment that is a wearable device and CO₂-sensing film for continuous transcutaneous monitoring of pCO₂. The film emission is excited via two (e.g. 405 nm and 470 nm) high-intensity LED's and sampled via a long-pass filter (e.g. 500 nm) and a PIN photodiode as a photodetector.

FIG. 1B shows optical spectra of the two different excitation LEDs and the CO₂-sensing dye emission, as shown in the inset. The addition of a 500 nm long-pass filter removes the LED emission.

FIG. 2 shows in panel (a), chemical structures of ion pairs and polymer matrices: (HPTS)/(CTA)₃, (HPTS)/(TOA)₄, poly (methyl methacrylate) (PMMA), and poly (propyl methacrylate) (PPMA). The pH sensitivity of the ion pairs arises from the highlighted functional groups. Panel (b) shows emission spectra of (HPTS)/(CTA)₃ in PPMA, and (HPTS)/(TOA)₄ in PPMA and PMMA under CO₂ and N₂ conditions. Panel (c) shows excitation spectra (collected at 570 nm) of (HPTS)/(CTA)₃ in PPMA, and (HPTS)/(TOA)₄ in PPMA and PMMA under CO₂ and N₂ conditions. Panel (d) shows moisture sensitivity of (HPTS)/(TOA)₄ in PPMA, and PMMA under CO₂ and N₂ conditions. Panel (e) shows photostability comparison of (HPTS)/(TOA)₄ in PPMA and PMMA under air condition.

FIG. 3 shows in panel (a), sensitivity of materials made of 240 μM or 480μM (HPTS)/(TOA)₄ in PPMA with the addition of 0%, 5%, 10%, 20%, and 40% (v/V) methanolic solution of TOAOH. Panel (b) shows photostability comparison of sensing films prepared from 240 μM (HPTS)/(TOA)₄ in PPMA containing 5%, 10%, and 20% (v/v) TOAOH solution and 480 μM (HPTS)/(TOA) 4 in PPMA with 20% (v/V) TOAOH solution under the air condition. Panel (c) shows moisture sensitivity of the material prepared from 240 μM (HPTS)/(TOA)₄ in PPMA with 10% (v/v) TOAOH solution under CO₂ and N₂ conditions.

FIG. 4 shows in panel (a), excitation spectra measured at 570 nm of the (HPTS)/(TOA)₄ in the PPMA formulation exposed to different CO₂ partial pressures. Panel (b) shows normalized R (between [0,1]) vs. CO₂ partial pressure of a PPMA/white coating sample, showing a delayed diffusion of CO₂ through the white coating (CO₂→white), which disappears at temperatures over 40° C. The fluorescence ratio R measured with the wearable device is highly sensitive to changes in CO₂, with our prototypes showing a delayed response with respect to the reference CO₂ sensor at panel (c) T=25° C., attributed to CO₂ diffusion through the white light scattering layer, vanishing when heating up to panel (d) T=44° C. Panel (e) shows normalized R vs. CO₂ for the wearable at different temperatures, with the delayed response vanishing at higher temperatures. Panel (f) shows time delay (lag) between our prototype's signal and the reference CO₂ sensor as a function of temperature.

FIG. 5 shows in panel (a), response of the film to changes in CO₂, plotted along with a reference sensor's CO₂ readings. Panel (b) shows fit of two different calibration algorithms to the fluorescence ratio R, plotted as a function of the reference CO₂. Fit1 considers a quadratic dependence on CO₂, while Fit2 also considers a quadratic dependence on CO₂, but with different coefficients depending on whether R (and hence, CO₂) is increasing or decreasing. Panel (c) shows reference and estimated CO₂ from our prototype, obtained with both algorithms.

FIG. 6 shows material aging and sensitivity, specifically the effect of aging time on sensitivity under ambient, dark condition for materials made of (HPTS)/(TOA)₄ in PPMA with 5% and 10% TOAOH.

FIG. 7 shows a materials spectral characterization, specifically spectra of HTPS (TOA)₄/PPMA taken with the reference FLS1000 photoluminescence spectrometer (Edinburgh Instruments Ltd), excited at 405 and 470 nm, with and without the 495 long pass filter.

FIG. 8 shows a materials spectral characterization, specifically spectra measured from the wearable device, compared to the spectra from the reference spectrometer from FIG. 7 (gray lines). Panel (a) shows 405 nm LED shows an unwanted phosphorescence which is filtered out using short-pass, flexible filters (SP) which allow only the LED emission necessary to excite the HPTS dye to transmit through. The LED emission is filtered before reaching the photodiode by a long-pass filter, so that only the HPTS emission is detected. Panel (b) shows the 470 nm LED does not require such filtering.

FIG. 9 shows a calibration setup, including a sealed chamber with heater 910 and reference CO₂ sensor 920, a CO₂ tank 950, a N₂ tank 940, and an automated gas mixer 930.

FIG. 10 shows the performance of the calibration algorithms, specifically residuals from panel (a) Fit1 and panel (b) Fit2. It can be clearly seen how Fit2 describes the data with higher fidelity, as the residual is randomly scattered, compared to Fit1, in which the trend of the data can still be detected. The first calibration algorithm, Fit1, is obtained by combining different kinetic rates and material parameters such as quantum yield, etc. Although this model captures the average trend of the response, the estimated pCO₂ is always either over-or under-estimated due to the different rates for protonation/de-protonation. To account for this, a second model is proposed, Fit2, which uses the same algorithm as Fit1, but with different calibration parameters depending on whether CO₂ increases or decreases.

FIG. 11 shows the performance of the calibration algorithms, specifically in panel (a), ideal vs. calibration model sensor response. Panel (b) shows a Bland-Altman plot for Fit1. Panel (c) shows a Bland-Altman plot for Fit2. The standard deviation of Fit2 is roughly half of Fit1.

FIG. 12 shows a linear calibration algorithm, specifically fitting of a linear model to the luminescence ratio vs. CO₂, as in Zhu et al., “A new ratiometric, planar fluorosensor for measuring high resolution, two-dimensional pCO₂ distributions in marine sediments”, Marine Chemistry 2006, 101, 40-53, which does not fully capture the curvature of the data.

Like reference numerals will be used to refer to like parts from Figure to Figure in the following detailed description. Any drawings herein are not shown to scale. Where dimensions are given in the text or figures, these dimensions are merely example values which could be used with one or more example implementations and do not limit the scope of the disclosed invention.

### DETAILED DESCRIPTION OF THE INVENTION

The present disclosure provides devices, materials, and methods for analyte monitoring, and more particularly, devices, materials, and methods for luminescence-based carbon dioxide sensing.

Before the present invention is described in further detail, it is to be understood that the invention is not limited to the particular embodiments described. It is also to be understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

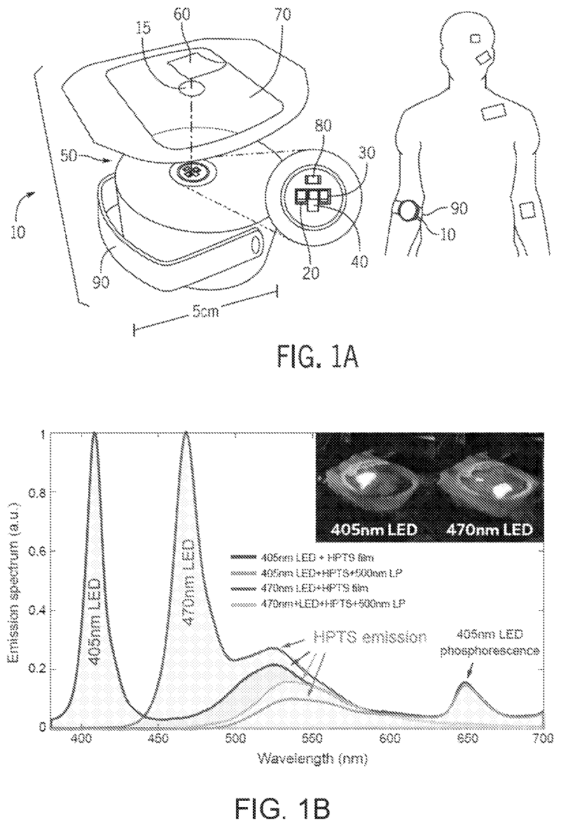

Referring now to FIG. 1A, there is shown one non-limiting example embodiment of a wearable device 10 according to the invention for transcutaneous carbon dioxide monitoring. The device 10 includes a photoluminescent carbon dioxide-sensitive probe 15 comprising a polymer matrix and a sensing dye. A first photon source 20 is configured to direct photons at a first wavelength at the probe 15, and a second photon source 30 is configured to direct photons at a second wavelength at the probe 15. A photodetector 40 is configured to detect light emitted from the probe 15 when the first photon source 20 and the second photon source 30 direct photons at the probe 15. Illumination from the photon sources can be modulated (e.g. square, sinusoidal, triangular, sawtooth, etc.) or constant. The photodetector 40 can be a PIN photodiode. A controller 50 is in electrical communication with the first photon source 20 and the second photon source 30 and the photodetector 40. The controller 50 is configured to execute a program stored in the controller 50 to calculate a level of carbon dioxide adjacent the probe 15 from an electrical signal received from the photodetector 40. The device 10 includes a carbon dioxide permeable light scattering layer 60, and a transparent semi-permeable adhesive film 70. The device 10 can include a heater and/or thermistor 80, and can also include a band 90 structured for securing the device 10 to a body part of a patient as shown in FIG. 1A. Since the device 10 can directly measure pCO₂, it does not require perfusion or underlying blood vessels. The sensing components can be safely retained within the device 10, which can be non-invasive, and as such, exogenous dyes, injectable agents, and needles may not be required. The optical device 10 can require low preparation time, and a readout of the device 10 can be essentially instantaneous. With the optical device 10, bedside calibration and heating may not be required, underlying blood flow may not be needed, and minimal equilibration time before getting results that can be presented on a simple readout, thereby providing a solution to the problems that plague clinical carbon dioxide sensing as a whole.

In the device 10, the photoluminescent carbon dioxide-sensitive probe 15 can comprise a polymer matrix. In some embodiments, the polymer matrix comprises a hydrophobic polymer. In some embodiments, the polymer matrix comprises a polymer selected from the group consisting of acrylate polymers and methacrylate polymers and blends and copolymers thereof. Non-limiting example acrylate polymers include poly (acrylic acid), poly (methyl acrylate), poly (ethyl acrylate), poly (propyl acrylate), and poly (butyl acrylate). Non-limiting example methacrylate polymers include poly (methyl methacrylate), poly (ethyl methacrylate), poly (propyl methacrylate), poly (butyl methacrylate), and poly (hydroxyethyl methacrylate). In some embodiments, the polymer matrix comprises a polymer selected from the group consisting of alkyl methacrylate polymers. In some embodiments, the polymer matrix comprises poly (propyl methacrylate) (PPMA).

In the device 10, the photoluminescent carbon dioxide-sensitive probe 15 can comprise a glassy material. In some embodiments, the glassy material can comprise a silicate sol-gel. Non limiting example silicate materials include orthosilicate, tetraalkyl orthosilicate, tetramethyl orthosilicate, tetraethyl orthosilicate, tetrapropyl orthosilicate, and tetrabutyl orthosilicate.

In the device 10, the photoluminescent carbon dioxide-sensitive probe 15 can comprise a polyurethane material. In some embodiments, the polyurethane material comprises a breathable film. In some embodiments, the polyurethane material comprises a breathable foam. In some embodiments, the polyurethane material can be composed from diisocyanate and polyol units. In some embodiments, the diisocyanate monomer can comprise an arylene diisocyanate. In some embodiments, the diisocyanate monomer can comprise an alkylene diisocyanate. Non-limiting example diisocyanate monomers include phenylene diisocyanate, methylene diphenyl diisocyanate, hexamethylene diisocyanate and dodecamethylene diisocyanate. In some embodiments, the polyol monomer can comprise an alkanoyl glycerol. Non-limiting example polyol monomers include octanoyl-glycerol and decanoyl-glycerol.

In the device 10, the photoluminescent carbon dioxide-sensitive probe 15 can comprise a sensing dye wherein a luminescence response of the sensing dye provides tissue pCO₂. In some embodiments, the sensing dye comprises a porphyrin molecule. In some embodiments, the sensing dye comprises a pH-sensitive fluorescent dye. In some embodiments, the pH-sensitive fluorescent dye comprises 8-hydroxy-1,3,6-pyrenetrisulfonic acid trisodium salt (HPTS). In some embodiments, the sensing dye comprises a pH-sensitive fluorescent dye in anion form and a quaternary ammonium cation. In some embodiments, the carbon dioxide-sensitive probe comprises a phase transfer reagent co-embedded with the sensing dye within the polymer matrix. In some embodiments, the phase transfer reagent has a volumetric ratio (v/v) with respect to the sensing dye of greater than 0% to 40%, or greater than 0% to 20%, or greater than 0% to 10%, or 2% to 8%. The phase transfer reagent provides water required for production of carbonic acid to protonate the pH-sensitive fluorescent dye. In some embodiments, the phase transfer reagent comprises an alkylammonium hydroxide. In some embodiments, the phase transfer reagent comprises tetraoctylammonium hydroxide. In some embodiments, the carbon dioxide-sensitive probe has a storage stability such that no significant decrease of sensitivity when the carbon dioxide-sensitive probe is stored under ambient and dark conditions for seven days. Light emitted from the probe 15 can be filtered by a long-pass filter covering the photodetector 40.

In some embodiments, the first photon source 20 can be a light-emitting diode emitting light having a first peak wavelength (e.g., 405 nanometers). In some embodiments, the second photon source 30 can be a light-emitting diode emitting light having a second peak wavelength (e.g., 470 nanometers). The photons from the photon sources 20, 30 can be filtered using a long-pass filter.

The controller 50 can be a microcontroller, or system-on-a-chip, and can comprise a memory which can be a non-transitory memory that can store executable programs on the controller 50. In some embodiments, the controller 50 can store an analyte (e.g., carbon dioxide) calculation program that can calculate a level of analyte (e.g., carbon dioxide) adjacent the probe 15 from one or more electrical signals received from the photodetector 40. The controller 50 can also include an output, which can be a wire bundle. The output can connect to an external interface which can be used for at least one of displaying, storing, and analyzing the results of the executable program of the device 10. The device 10 may include a display in electrical communication with the controller 50, wherein the controller is configured to execute the program stored in the controller to display the level of carbon dioxide on the display and/or store values of the level of carbon dioxide at a plurality of times over a time period. In other embodiments, the controller 50 can be configured to have a wireless output; the wireless output can perform wireless communication. Non-limiting examples of wireless communication that can be incorporated are Wi-Fi, Bluetooth®, near-field communication, cellular network, radiofrequency, etc. The controller 50 can comprise an onboard power source, for example a battery, that can provide power to the controller 50 such that the emission sources 20, 30, the photodetector 40, and the controller 50 can be powered. In other embodiments, the controller 50 can comprise an external power source, for example electrical connection to grid power, such that the emission sources 20, 30, the photodetector 40, and the controller 50 can be powered.

In the device 10, a carbon dioxide permeable light scattering layer 60 can be present, and can comprise a silicone film including a pigment. The pigment can be white. The light scattering layer 60 allows for the fluorescence emission to be backscattered to the photodetector 40. The white light scattering layer 60 also serves as an optical insulation, preventing external lighting from affecting the measurement, yielding a reading that is independent of skin tone. The heater and/or thermistor 80 can increase and/or measure a temperature of the light scattering layer 60. In the device 10, the transparent semi-permeable adhesive film (e.g., air-impermeable) layer 70 can be used to seal out room air from the probe 15 and light scattering layer 60, allowing the material to equilibrate to skin pCO₂. The device 10 for transcutaneous CO₂ sensing is biocompatible since only the multilayer film makes contact with the skin. Of the total area of the film, close to 99% corresponds to a commercial, medical-grade, skin adhesive of layer 70 and the remaining 1% to the light scattering layer 60, which is compatible with skin and can even be inserted into tissue. The probe 15 is prevented from direct contact with the skin via the light scattering layer 60.

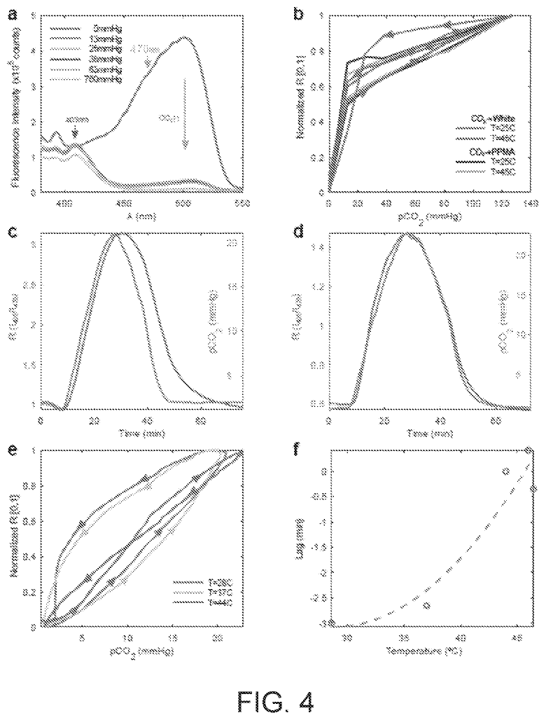

Now that the components of the device 10 have been described in detail, the method of operating the optical device 10 can be understood. While the device 10 is especially useful in detecting a concentration of carbon dioxide, the device 10 can be used in a method for detecting a concentration of other analytes. In a non-limiting example method of operating the optical device 10, the first photon source 20 is activated by the controller 50 to direct photons at a first wavelength at the probe 15, and the second photon source 30 is activated by the controller 50 to direct photons at a second wavelength at the probe 15, wherein the second wavelength is different from the first wavelength. The photodetector 40 detects light emitted from the probe 15 when the first photon source 20 and the second photon source 30 direct photons at the probe 15. The controller 50 executes a program stored in the controller 50 to calculate a concentration of the analyte adjacent the probe 15 based on the light emitted from the probe detected by the photodetector 40. An isosbestic point in the excitation spectra of the sensing dye can be used advantageously in the detection of the analyte as demonstrated in FIG. 4a.

The controller 50 can execute the program stored in the controller 50 to calculate the concentration of the analyte adjacent the probe 15 based on an algorithm, calibration, and/or normalization factor determined by directing photons at the first wavelength at the probe to account for variations in brightness of the polymer matrix. The controller 50 can execute the program stored in the controller 50 to calculate the concentration of the analyte adjacent the probe based on a luminescence (e.g., fluorescence) ratio providing a metric that is proportional to the concentration of analyte adjacent the probe and is normalized using the normalization factor. The controller 50 can execute the program stored in the controller 50 to calculate the concentration of the analyte adjacent the probe based on a calibration algorithm such that the device can report the concentration of analyte adjacent the probe via wired or wireless communication (e.g., USB or Wi-Fi). The calibration algorithm may use different calibration parameters depending on whether a derivative of the luminescence (e.g., fluorescence) ratio is positive or negative. The controller 50 can execute the program stored in the controller 50 to calculate the concentration of the analyte adjacent the probe by integrating emission spectra excited at the first wavelength and the second wavelength. The controller 50 can execute the program stored in the controller 50 to calculate the concentration of the analyte adjacent the probe by modulating by a sinusoidal voltage and an intensity of a luminescence (e.g., fluorescence) response of the sensing dye is defined as an amplitude of a measured sinusoidal response, which is extracted via multiple linear regression. In some embodiments, the analyte can be selected from the group consisting of molecular oxygen, carbon dioxide, nitric oxides, dissolved analytes in plasma and tissue, and hydrogen ions. In some embodiments, the analyte can be selected from the group consisting of molecular oxygen and carbon dioxide and mixtures thereof. In some embodiments, the analyte is carbon dioxide, the concentration of the analyte is calculated as pCO₂, and the physiological range of pCO₂ can be 0-50 mmHg.

### EXAMPLE

The following Example is provided to demonstrate and further illustrate certain embodiments and aspects of the present invention and is not to be construed as limiting the scope of the invention. The statements provided in the Example are presented without being bound by theory.

### Overview of Example

Continuously monitoring CO₂ partial pressure is of crucial importance in the diagnosis and treatment of respiratory and cardiac diseases. Despite significant progress in the development of CO₂ sensors, their implementation as portable or wearable devices for real-time monitoring remains under-explored. In this Example, we report on the creation of a wearable prototype device for transcutaneous CO₂ monitoring based on quantifying the fluorescence of a highly breathable CO₂-sensing film. The developed materials are based on a fluorescent pH indicator (8-hydroxy-1,3,6-pyrenetrisulfonic acid trisodium salt or HPTS) embedded into hydrophobic polymer matrices. The film's fluorescence is highly sensitive to changes in CO₂ partial pressure in the physiological range, as well as photostable and insensitive to humidity. The device and medical-grade films are based on our prior work on transcutaneous oxygen-sensing technology, which has been extensively validated clinically.

### 1 Introduction

Carbon dioxide (CO₂) plays various roles in the human body such as the respiratory drive, regulation of blood pH, and affinity of hemoglobin for oxygen [Ref. 1]. Monitoring CO₂ partial pressure (pCO₂) in breath and blood is of great importance for both the medical diagnosis and treatment of human diseases such as respiratory and metabolic disorders. For example, the adequacy of spontaneous and mechanical ventilation is usually evaluated by measuring the CO₂ concentration in arterial blood [Ref. 2, 3]. However, the current “gold standard” method to obtain this reading relies on the invasive process of arterial blood gas sampling through the placement of arterial lines [Ref. 2, 3]. Although there exist other non-invasive, albeit indirect, clinical alternatives for CO₂ sensing, these techniques require further development and optimization to improve accuracy, integration into clinical workflows, etc., as they exhibit important limitations or drawbacks. For example, end-tidal CO₂ sensors use an infrared absorption technique for measuring the level of CO₂ released at the end of an exhaled breath [Ref. 4]. However, this technique requires the use of an optical cavity, requiring large working volumes of gas compared to other methods [Ref. 5], and hence, its accuracy to estimate arterial pCO₂ is susceptible to sampling errors and patient-related factors (e.g., age, patient positioning, lung disease) [Ref. 6, 7].

A non-invasive alternative to arterial gas sampling is to monitor pCO₂ on the skin surface, which can reduce or altogether eliminate the need for blood gas sampling, decreasing the risk for patient co-morbidities and improving patient comfort. Transcutaneous monitoring of CO₂ can be critical, for example, to assess ventilation in neonates [Ref. 8], for which periodic arterial blood sampling can be painful and does not provide continuous readings, and monitoring end-tidal CO₂ is not possible due to the small tidal volumes. There are many different locations on the body that can be used for transcutaneous monitoring, depending on the clinical scenario, on which transcutaneous pCO₂ is highly correlated to arterial pCO₂, typically highly vascularized areas with thin skin. Some examples include the earlobe [Ref. 9], the neck near the carotid artery [Ref. 10], the lateral abdomen, the anterior or lateral chest [Ref. 11], the volar forearm, the inner upper arm or the inner thigh [Ref. 12], the foot [Ref. 13], etc. However, current technology based on electrochemical sensors requires large and expensive equipment, long bed-side calibration procedures, immobile patients, etc. [Ref. 3,14]. Therefore, it would be of considerable interest to develop miniaturized sensors that could be incorporated into wearable devices and find wide application, such as the continuous measurement of transcutaneous CO₂.

Optical transcutaneous CO₂ sensors based on luminescent materials may offer several advantages, such as accurate detection of CO₂ levels, as well as great potential for miniaturization [Ref. 15]. Such sensors have traditionally employed a pH indicator that exhibits different fluorescent intensities upon exposure to different CO₂ concentrations [Ref. 16-18]. The pH-sensitive fluorescent dye 8-hydroxy-1,3,6-pyrenetrisulfonic acid trisodium salt (HPTS) is one of the most widely used in optical CO₂ sensors [Ref. 19, 20]. For skin-worn devices, it can be important to create sensors whose response will not be altered by changes in humidity [Ref. 21, 22], which can vary widely depending on climate or body location [Ref. 23]. In order to make HPTS molecules compatible with hydrophobic matrices, a lipophilic hydrated ion pair is usually formed by converting the dye into its anion form with a quaternary ammonium cation [Ref. 16,17,19]. In addition, a phase transfer reagent (quaternary ammonium hydroxide) co-embedded along with the dye within a support matrix is necessary to facilitate the tuning of the materials' sensitivity and enhanced stability. Therefore, the performance of the CO₂ sensors depends not only on the properties of the dye molecule, but also on the optical and physical properties of the support matrix.

In this Example, we report on the development of polymer films with embedded HPTS-based ion pairs, providing highly breathable materials that exhibit bright emission throughout the physiological CO₂ range. We created a wireless and non-invasive wearable prototype that, in conjunction with the proposed materials, aims to continuously monitor transcutaneous CO₂ partial pressure.

### 2. Material and Methods

### 2.1. Materials

HPTS, tetraoctylammonium bromide (TOABr), tetraoctylammonium hydroxide solution (20% in methanol) (TOAOH), hexadecyltrimethylammonium hydroxide solution (25% in methanol) (CTAOH), poly (methyl methacrylate) (PMMA) (approx. Mw 75,000), platinum (0)-1,3-divinyl-1,1,3,3-tetramethyldisiloxane complex solution, and sodium sulfate were purchased from Sigma-Aldrich. Cetyltrimethylammonium bromide (CTABr) was purchased from Fisher Scientific. Poly (propyl methacrylate) (PPMA) was purchased from Scientific Polymer Products (approx. Mw 150,000). The white pigment concentrate, (45-55% methylhydrosiloxane)-dimethylsiloxane copolymer (HMS), and cure-retarding agent were purchased from Gelest. Glass microfiber filters were purchased from Whatman and the medical-grade adhesive films (Bioclusive) from Mckesson.

### 2.2. Synthesis of Ion Pairs

### 2.2.1. Synthesis of (HPTS)/(CTA)₃

This compound was synthesized by ion-pairing HPTS with CTABr, which was similar to the approach adopted by Burke et al. [Ref. 24]. Eighty milligrams of CTABr was dissolved in 5 mL of ultrapure water at 50° C. and mixed with a solution comprising 40 mg of HPTS in 5 mL of ultrapure water. The product was obtained by vacuum filtration followed by washing with ultrapure water. The solid product was dried in an oven at 50° C. for 1 hour.

### 2.2.2. Synthesis of (HPTS)/(TOA)₄

The (HPTS)/(TOA)₄ was prepared by the following method [Ref. 25]: 20 mg of HPTS and 4-fold molar equivalents of TOABr (85 mg) were dissolved in 5 mL of 0.01 M NaOH solution and 5 mL of dichloromethane, respectively. The two solutions were subsequently mixed together, and the reaction mixture was stirred for about 1 hour at room temperature. The mixture was added into a separatory funnel, and the ion pair was extracted into the organic layer followed by washing twice with 5 mL 0.01 M NaOH solution. The organic layer was collected and dried from traces of water over sodium sulfate. The solvent was removed by rotary evaporation, and the solid product was dried under high vacuum. The product yield was calculated to be about 60%.

### 2.3. CO₂-Sensing Film Preparation

### 2.3.1. Films for Spectral Characterization

Filter paper was used as the substrate material to produce samples for spectral characterization as it is highly breathable and provides light scattering, enhancing the amount of collected light by our spectrometer. An aliquot of ion pair solution was added into 0.05 mg/μL PMMA or PPMA in dichloromethane followed by different ratios of methanolic CTAOH or TOAOH and mixed thoroughly by vortexing. Then, 60 μM (HPTS)/(CTA)₃ and 240 or 480 μM (HPTS)/(TOA)₄ were applied to the final solutions. Approximately 15 μL of that solution was deposited onto the 5 mm-diameter filter paper, which was placed on a solid surface and was left to dry in the hood overnight at room temperature.

### 2.3.2. Multilayer CO₂ Sensing Film for the Wearable

As shown in FIGS. 1A and 1B, the multi-layer CO₂-sensing film was fabricated following the approach in [Ref. 26], by stacking a breathable and white (scattering) silicone film as a carbon dioxide permeable light scattering layer 60, a PPMA-based sensing film as a photoluminescent carbon dioxide-sensitive probe 15, and a transparent semi-permeable adhesive film (Bioclusive, Mckesson, New York, NY, USA) 70. This configuration was used along with the wearable device due to its reduced volume and thickness and, therefore, fast equilibration to the skin CO₂ concentration. The combination of the white silicone layer 60/PPMA layer 15 was used in place of filter paper in order to minimize the dead-space volume while allowing for the fluorescence emission to be backscattered to the wearable device's photodiode 40. The white coating also serves as an optical insulation, preventing external lighting from affecting the measurement, yielding a reading that is independent of skin tone. The semi-permeable, optically transparent adhesive film 70 was used to seal out room air from the CO2-sensing film and white silicone layer 60, allowing the material to equilibrate to skin pCO₂. Our approach to transcutaneous CO₂ sensing is biocompatible since only the multilayer film makes contact with the skin. Of the total area of the film, close to 99% corresponds to a commercial, medical-grade, skin adhesive and the remaining 1% to the white silicone coating, which is compatible with skin [Ref. 26] and can even be inserted into tissue [Ref. 27]. The HPTS/PPMA disk 15 is prevented from direct contact with the skin via the white coating 60.

To prepare the PPMA-based film, a 25 μL solution of PPMA with (HPTS)/(TOA)₄ and TOAOH was deposited into an 8 mm-diameter circular mold on a glass slide. The PPMA-based film was removed from the glass slide after drying in the hood for 30 minutes. The white light scattering layer was prepared following the protocol in [Ref. 26] with the following modifications. The silicone polymer component (100 μL) was first mixed with the white pigment concentrate (1 g), and then, 3 drops of cure retarding agent and 1 drop of platinum catalyst were added. The mixture was deposited on a flat surface and was able to form a thin film before curing.

### 2.4. Principle of Operation

The most common principle for optical CO₂ sensors is based on fluorescence changes of pH indicators upon their protonation or deprotonation at different pH values [Ref. 17, 18]. In HPTS, it has been widely reported that the pH sensitivity of HPTS arises from the OH group [Ref. 19]. To facilitate the dissolution of the dye in the hydrophobic polymers, the lipophilic quaternary ammonium cation (Q⁺) (known as the phase transfer agent) is widely used to form ion pairs with the pH indicator anion (D⁻) and provides the water required for the production of carbonic acid to protonate the pH indicator dye [Ref. 16]. The general chemical principle can be described as follows:

Q⁺D⁻.xH₂O+CO₂=Q⁺HCO₃⁻.(x−1)H₂O+DH (1)

A number of water molecules are found to be associated with the ion pair when a phase transfer agent is used to extract an anion indicator from an aqueous solution into an organic solution [Ref. 18]. In the presence of CO₂, the dye anion with green fluorescence is converted into its protonated form, which does not fluoresce under 470 nm excitation. The reaction of the indicator dye with CO₂ is fully reversible. In addition, the quaternary ammonium hydroxide is introduced in order to tune the sensitivity of the materials.

### 2.5. Fluorescence Spectral Measurements

Fluorescence spectral measurements were acquired using an FLS1000 Steady State and Luminescence Lifetime Spectrometer equipped with a continuous xenon lamp (Xe2) (Edinburgh Instruments, Livingston, UK). The sample tested was placed on a holder affixed diagonally inside a cuvette with a septum screw cap. Changes in gas conditions were generated by flowing a N₂/CO₂ gas mixture via a needle through the septum of the cuvette cap. The gases were also connected to a water bubbler to modify the water vapor content of the mix. Excitation spectra were acquired by setting the emission wavelength at 570 nm and scanning the excitation wavelength from 300 nm to 550 nm. Emission spectra were acquired by setting the excitation wavelength at 405 or 470 nm and scanning the emission wavelength from 500 nm to 700 nm. For the emission spectra, the excitation light was filtered using a 495 nm long-pass filter for all the samples, which is consistent with the wearable's filters and therefore provides a “ground truth” measurement. The photostability of the different materials was measured using a Kinetic Scan with a fixed excitation wavelength at 470 nm and a fixed emission wavelength at 520 nm for a time period of 120 minutes under air conditions. The power of the excitation light that the samples were exposed to was set by adjusting the excitation bandwidth and was measured by an optical power meter (Thorlabs, PM100D, Newton, NJ, USA). The percent change of the intensity was calculated as follows:

$$I \% = \frac{I - {I}_{min}}{{I}_{max}} \cdot 100 \qquad ( 2 )$$

### 2.6. Wearable Optical Device

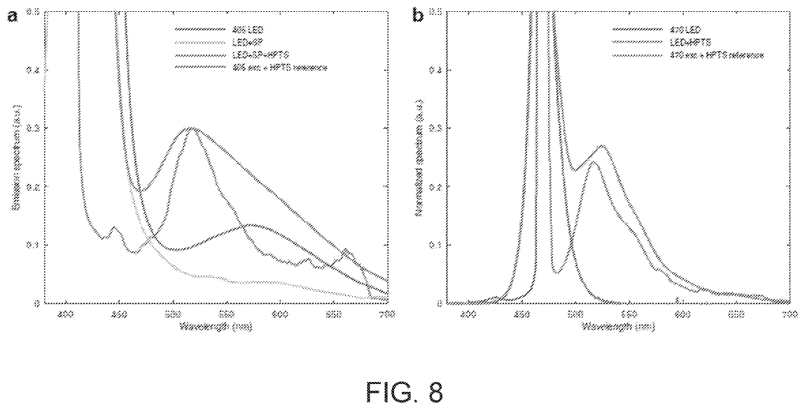

Based on our previous efforts [Ref. 26], we developed a small and lightweight wearable prototype (see FIG. 1A), which can provide readings of the partial pressure of CO₂ by exciting and detecting the fluorescence response of the CO₂-sensing films described above. The devices are based on a Wi-Fi-enabled microcontroller board (Particle Photon), which drives the low-power, custom electronics (a fast ADC chip, transimpedance amplifier, and signal-conditioning block). The intensity of the fluorescence response of the film is excited sequentially by two high-power LEDs with peak wavelengths of 405 and 470 nm (see FIG. 1B) and detected via a PIN photodiode. The LEDs are modulated by a sinusoidal voltage signal of f=1.6 kHz, and the intensity of the emission from the CO2-sensing film excited by each of the LEDs is defined as the amplitude of the measured sinusoidal response, which is extracted via multiple linear regression [Ref. 26]. The temperature of the film and sensor head was sampled through a small thermistor. To avoid cross-talk between excitation and emission and to remove an unwanted phosphorescence from the LED's (see FIG. 8), the LED excitation was filtered by a 500 nm short-pass filter composed of two ultra-thin flexible optical notch filters (Edmund Optics), which is in turn were blocked by a 500 nm long-pass filter covering the PIN photodiode, by combining a flexible 405 nm long-pass filter (Edmund Optics) and an “amber” color polyamide film (Kapton® tape, 3M). The CO₂ sensing films were attached onto the device's 3D-printed casing as a support structure using thin, highly adhesive double-sided tape. The device adheres to the skin via the medical-grade film and an elastic band or strap, which is minimally tightened to prevent the restriction of blood flow. Data were collected via a Python [Ref. 28] script on a PC through a USB serial port in order to record the sinusoidal time series each time CO₂ is sampled. For calibrated devices, the calibration algorithm (described below) can be loaded onto the firmware, so the devices directly report CO₂ readings via USB or Wi-Fi to a display.

### 3. Results and Discussion

### 3.1. Optimization of Sensing Film Compositions

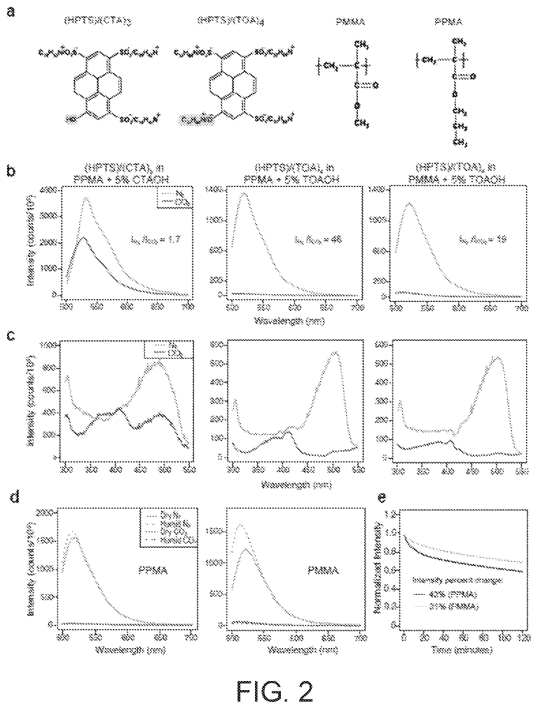

To test the effect of different dyes and polymer matrices on the sensitivity of the CO₂ sensors, the following materials cast on filter paper were investigated: (1) (HPTS)/(CTA)₃ in PPMA; (2) (HPTS)/(TOA)₄ in PPMA; and (3) (HPTS)/(TOA)₄ in PMMA. The filter paper was used as a support material for spectral measurements, as it is highly light scattering and does not change the sensing properties of the materials. Fluorescence spectral changes upon excitation at 470 nm are shown in FIG. 2, panel b. The ratio of I_{N2}/I_{CO2} was used as an indicator of the sensitivity of the CO₂ sensors, where I_{N2} and I_{CO2} correspond to the peak intensities of the spectra when the materials are exposed to a 100% N₂ and a 100% CO₂ environment, respectively. It was found that the I_{N2} of (HPTS)/(CTA)₃ embedded in PPMA was about 1.7-fold stronger than the I_{CO2}. The CO₂ sensitivity dramatically increased to about 46 when the sensing molecule was replaced by the (HPTS)/(TOA)₄. In addition, it was observed that when (HPTS)/(TOA)₄ embedded in PMMA, I_{N2} was about 19-fold stronger than I_{CO2}. These results demonstrated that the sensitivity of the CO₂ sensors could be tuned by adjusting the composition of the sensing molecules and the support matrices.

As has been previously shown for sensors embedded within similar types of synthetic acrylate polymer matrices [Ref. 22], excitation spectra can provide useful information on the distribution of the embedded sensor molecules and the homogeneity of the resulting materials. Chemical compatibility between the embedded dyes and polymer matrix components featuring hydrophobic functional groups and alkyl chains can have a major impact on sensing performance [Ref. 21,22]. To gain more insight into the differences in sensitivity observed among the materials tested here that showed (HPTS)/(TOA)₄+PPMA exhibiting the highest sensitivity, excitation spectra were acquired and presented in FIG. 2, panel c. For all three materials, the dye exhibited maximum excitation at about 400 nm and 500 nm under CO₂ and N₂ conditions, respectively. The different excitation wavelengths represent the protonated or deprotonated forms of the dye in the presence of CO₂ or N₂. It was found that (HPTS)/(CTA)₃ in PPMA along with CTAOH was only partially converted to the protonated form under the CO₂ condition, which explains the low CO₂ sensitivity of this material. In contrast, (HPTS)/(TOA)₄ embedded in PPMA along with TOAOH was almost completely converted to the protonated form under the CO₂ condition, which could result in the observed much higher sensitivity. In addition, it can be clearly seen that the excitation spectrum of (HPTS)/(CTA)₃ in PPMA along with CTAOH is much broader than that of (HPTS)/(TOA)₄ in PPMA along with TOAOH under both the CO₂ and N₂ conditions. It is worth noting that, while CTAOH bears a single C₁₆ alkyl chain, TOAOH has four octyl (C₈) chains, which make it overall more lipophilic. Taken together, these observations suggest there is a poor homogeneity of (HPTS)/(CTA)₃ in PPMA along with CTAOH, likely leading to effects such as sensor ion pair aggregation, while the most optimally performing material is a combination of the more lipophilic quaternary ammonium ion and phase transfer reagent (TOA⁺/TOAOH) along with the polymer matrix with the longer and more hydrophobic side chain (PPMA). Moreover, it is likely that the relatively longer alkyl side chains of PPMA increase the diffusion and solubility of CO₂ gas in the matrix, which allows the dye to be protonated more efficiently and ultimately increases the CO₂ sensitivity, as has been previously noted [Ref. 29].

Since (HPTS)/(CTA)₃ in PPMA along with CTAOH showed much lower sensitivity, subsequent characterizations were only focused on the other two materials. Due to the water-mediated sensing chemistry, the moisture levels of the gas potentially have a strong influence on the sensor performance, particularly in applications such as respiratory gas analysis [Ref. 16]. Therefore, the CO₂ sensor performance was further assessed under humid conditions (FIG. 2, panel d). It was found that, while the presence of humidity did not affect the sensitivity of (HPTS)/(TOA)₄ in PPMA+5% TOAOH towards CO₂ sensing, the corresponding sensitivity for (HPTS)/(TOA)₄ in PMMA+5% TOAOH increased by 1.9-fold. It is possible that the longer alkyl side chains of PPMA make it more hydrophobic and better resistant to water when compared to PMMA. Lastly, although the PMMA-based material showed better photostability during two hours of continuous illumination (FIG. 2, panel e), higher gas sensitivity and better water resistance make (HPTS)/(TOA)₄ in PPMA an optimal candidate for final characterization.

### 3.2. The Effect of TOAOH Ratios on Sensitivity, Photostability, and Dark Stability

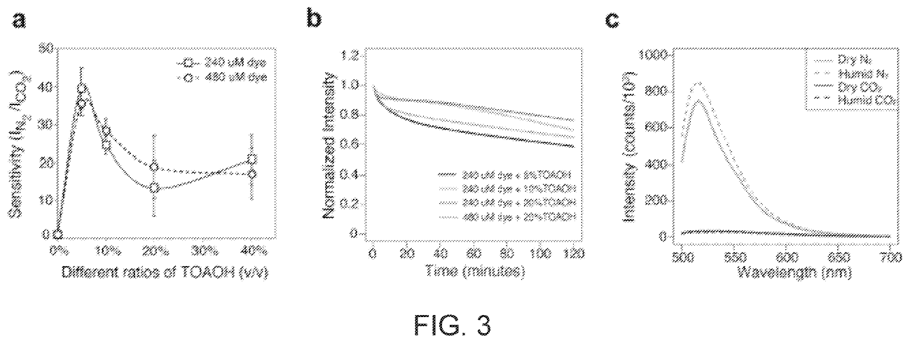

After identifying the optimal combination of polymer matrix and sensing dye, the effect of different amounts of TOAOH on the CO₂ sensing performance was investigated (see FIG. 3). TOAOH is beneficial for CO₂ sensing, as films prepared from the solution without this base buffer yielded no sensitivity to CO₂. It was also found that (HPTS)/(TOA)₄ in PPMA exhibited the highest sensitivity with the addition of a 5% (v/v) methanolic solution of TOAOH. In addition, the CO₂ sensitivity decreased with the further increase of the TOAOH ratios. A similar trend was observed when a different dye concentration was used (FIG. 3, panel a).

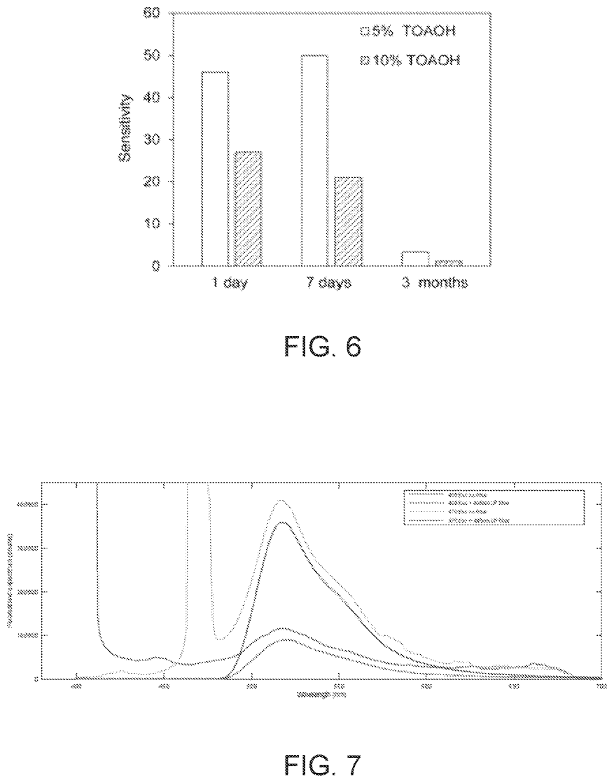

To characterize the photostability of the materials, the percentage changes in intensity were obtained after continuous irradiation for 120 minutes in air. This illumination time represents potentially days or weeks of measurement time with the wearable, depending on the sampling rate, as the acquisition of the CO₂ concentration lasts only 200 milliseconds per measurement. As shown in FIG. 3, panel b, (HPTS)/(TOA)₄ (240 μM) in PPMA showed higher photostability at higher TOAOH ratios. The percentage intensity changes were found to be 42%, 30%, and 24% for the sensing films made of 240 μM (HPTS)/(TOA)₄ in PPMA with the addition of 5%, 10%, and 20% (v/V) TOAOH solution. In addition, 480 μM dye was observed to be less photostable than 240 μM dye in the same support matrix. Although higher TOAOH ratios can improve the materials' photostability, it decreased the CO₂ sensitivity and also caused (HPTS)/(TOA)₄ in PPMA to become less water resistant. It was found that the CO₂ sensitivity increased by 1.4-fold under humid conditions with the addition of 10% (v/v) TOAOH (FIG. 3, panel c), whereas only 1.1-fold increased sensitivity was observed for 5% (v/v) addition of TOAOH (FIG. 2, panel d). The storage stability of (HPTS)/(TOA)₄ in PPMA with the addition of 5% and 10% TOAOH was tested over three months. No significant decrease of sensitivity was observed when the materials were stored under ambient (approximately 25° C.) and dark conditions for seven days. However, both materials lost about 90% sensitivity after three months (see FIG. 6). The stability of the materials could be further improved by optimizing the storage conditions. Therefore, considering its high CO₂ sensitivity and optimized water resistance and photostability, the formulation (HPTS)/(TOA)₄ in PPMA with 5% TOAOH was tested in combination with a wearable optical device.

### 3.3. Response and Calibration of the Wearable

As mentioned above, the wearable collects the emission after the dye molecules are excited at two different wavelengths, 405 nm and 470 nm, as was done in [Ref. 25]. The excitation spectra of the HPTS/TOA-PPMA formulation revealed an isosbestic point around 405 nm in the CO₂ range of interest (0-50 mmHg), as shown in FIG. 4, panel a. This wavelength is therefore an ideal reference or normalization factor to account for variations in film brightness, such as photobleaching, changes in relative positioning between film/device due to motion, etc. The second excitation wavelength used was 470 nm, which yielded a CO₂-dependent emission from the films. This wavelength was also chosen to allow for sufficient spectral separation between the excitation light (470 nm) and the dye's emission (520-530 nm) in order to reduce LED leakage into the photodiode. The use of two excitation wavelengths, with the excitation at 405 nm effectively acting as an isosbestic point in our CO₂ range of interest, allowed us to employ a ratiometric approach, which yielded a metric that was proportional to the CO₂ concentration and was normalized, not subject to photobleaching effects, and robust against motion artifacts within some range.

Therefore, we define the fluorescence ratio as:

R=I₄₀₅/I₄₇₀ (3)

As a gold-standard reference, we obtained the fluorescence spectra of TOA/HPTS in PPMA within the multilayer film structure, including a white light scattering layer. The measurements were taken by exciting at 405 and 470 nm, with the emission passed through a 495 nm long-pass filter to remove the excitation light from the collected light. To better compare spectrometer measurements with the wearable readings, I₄₀₅ and I₄₇₀ were calculated by integrating the spectra excited at 405 and 470nm, rather than defining these as the fluorescence intensity at a given wavelength. The integrated signal was smoother and more representative of the signal measured by the wearable's photodiode, which contains contributions from a wide range of wavelengths.

The definition of I_{405,470} as the integral of the fluorescence spectrum (with a long-pass filter to remove the excitation), and the ratio R=I_{405/}I₄₇₀ should be equivalent to the trends observed if I_{405,470} are measured at a single wavelength.

To explain this, we propose the following assumptions. Let's define the fluorescence spectrum excited by λ₁=405 nm and λ₂=470 nm as fλ₁(λ) and fλ₂(λ). As we can see in FIG. 2 and FIG. 3, the shape (or function with respect to wavelength) of the spectrum does not change with CO₂, and its amplitude scales proportionally with excitation intensity A_{exc}^{λi}∝I_{exc}^{λi} (also given the quantum yield) and, in the case of λ₂=470 nm, with CO₂ with some function B=h(CO₂), meaning:

$${f}_{{λ}_{1}} ( λ ) = {A}_{exc}^{{λ}_{1}} \cdot {g}_{{λ}_{1}} ( λ ) \\ {f}_{{λ}_{2}} ( λ ) = {A}_{exc}^{{λ}_{2}} \cdot B \cdot {g}_{{λ}_{2}} ( λ ) \\ so \\ {I}_{{λ}_{1}} = {\int }_{{λ}_{min}}^{{λ}_{max}} {f}_{{λ}_{1}} ( λ ) d λ = {\int }_{{λ}_{min}}^{{λ}_{max}} {A}_{exc}^{{λ}_{1}} \cdot {g}_{{λ}_{1}} ( λ ) d λ = {A}_{exc}^{{λ}_{1}} \cdot {C}_{{λ}_{1}} \\ {I}_{{λ}_{2}} = {\int }_{{λ}_{min}}^{{λ}_{max}} {f}_{{λ}_{2}} ( λ ) d λ = {\int }_{{λ}_{min}}^{{λ}_{max}} {A}_{exc}^{{λ}_{2}} \cdot B \cdot {g}_{{λ}_{2}} ( λ ) d λ = {A}_{exc}^{{λ}_{2}} \cdot B \cdot {C}_{{λ}_{2}} \\ with {A}_{exc}^{{λ}_{i}} and {C}_{{λ}_{i}} = {\int }_{{λ}_{min}}^{{λ}_{max}} {g}_{{λ}_{i}} ( λ ) d λ which do not depend on {CO}_{2} . \\ Therefore : \\ R = \frac{{I}_{{λ}_{1}}}{{I}_{{λ}_{2}}} = \frac{{A}_{exc}^{{λ}_{1}} \cdot {C}_{{λ}_{1}}}{{A}_{exc}^{{λ}_{2}} \cdot B \cdot {C}_{{λ}_{2}}}$$

and if the excitation source intensities, or their ratio (A_{exc}^{λ}^{₁}/A_{exc}^{λ}^{₂}=const) remain constant, and because the fluorescence spectra do not change shape, therefore the integrals C_{λ}, do not change, then the ratio changes proportionally to CO₂ through B=h(CO₂), i.e., R=K·B⁻¹ with K=(A_{exc}^{λ}^{₁}·C_{λ}_{₂})/(A_{exc}^{λ}^{₂}·C_{λ}_{₂})=constant.

If we do not integrate but pick a single wavelength, say λ₀=525 nm, then:

$${I}_{{λ}_{1}} ( {λ}_{0} ) = {A}_{exc}^{{λ}_{1}} \cdot {g}_{{λ}_{1}} ( {λ}_{0} ) \\ {I}_{{λ}_{2}} ( λ ) = {A}_{exc}^{{λ}_{2}} \cdot B \cdot {g}_{{λ}_{2}} ( {λ}_{0} ) \\ and \\ R = \frac{{I}_{{λ}_{1}}}{{I}_{{λ}_{2}}} = \frac{{A}_{exc}^{{λ}_{1}} \cdot {g}_{{λ}_{1}} ( {λ}_{0} )}{{A}_{exc}^{{λ}_{2}} \cdot B \cdot {g}_{{λ}_{2}} ( {λ}_{0} )}$$

which is equivalent to R when we integrate the spectrum, where the constants C_{λ}_{ᵢ} are replaced by g_{λ}_{ᵢ} (λ₀).

Additionally, calculating the ratio R using the integral value yields a smoother function compared to calculating it using the intensities at a single wavelength, as the integral provides an averaging of the spectrum.

FIG. 4, panel b plots the fluorescence ratio R as a function of the CO₂ concentration (calculated from the known mix of CO₂ and N₂ gases) in a PPMA/white coating sample in which CO₂ either diffused freely through PPMA (CO₂→PPMA) or was forced to do so through the white coating (CO₂→White). We allowed one minute for each gas mix to flow before the spectrum was taken. The ratio R was normalized between [0, 1] to better compare the trend at different temperatures. When CO₂ directly diffuses into the PPMA layer (CO₂→PPMA), the sample exhibits a cyclical response (start and end points match) that is independent of temperature. The small delay in response speed between increasing and decreasing CO2 has been previously reported [Ref. 25, 30] as being due to a difference in the reaction speed between protonation and de-protonation. The diffusion of CO₂ to the PPMA layer through the white coating (CO₂→White) was achieved by adding a transparent, but non-breathable backing layer to force all diffusion to occur through the white coating. As is shown in FIG. 4, panel b, the white coating adds a considerable delay in the diffusion of CO₂ out of the film at 25° C., not found to happen with oxygen [Ref. 26]. The breathability of the white film was greatly enhanced by increasing the temperature above 40° C., as is also seen in the wearable measurements in FIG. 4.

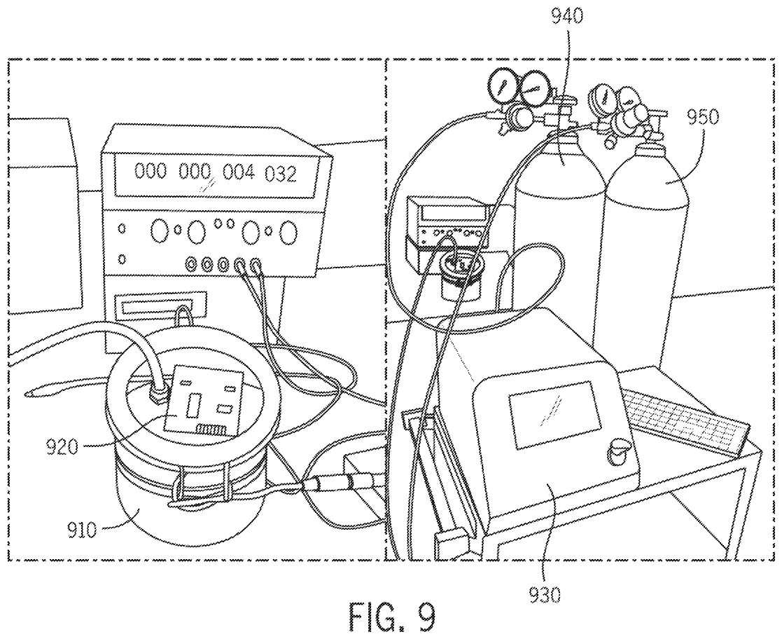

Measurements with the wearable device were carried out in a sealed chamber, where a controlled mix of N₂ and CO₂ gases was fed in using a programmable gas proportioner (Gometrics) as shown in FIG. 9. The pCO₂ in the chamber was measured with a commercial non-dispersive infrared (NDIR) CO₂ sensor (K-33 BLG, CO₂ Meter, Ormond Beach, FL, USA). We found a small amount of LED emission leaks through the optical filters and in the photodiode signal. We quantified this leakage as a function of temperature by measuring with the wearable a “blank” film, i.e., a multilayer film containing all the described layers, but no HPTS dye. This LED leakage was subtracted from all the I₄₀₅, I₄₇₀ values used to calculate R. FIG. 4, panels c,d shows the fluorescence ratio change due to the same gas mix sequence, measured at an average temperature of 25° C. and 44° C. The lower temperature was found to lag with decreasing CO₂, while the higher temperature showed no delay. Further, the normalized R vs. pCO₂ loops shown in FIG. 4, panel e, displayed the same trend with temperature as shown in the spectral measurements from FIG. 4, panel b. FIG. 4, panel f, plots the temperature dependence of the delay or lag between the wearable and the reference commercial sensor, obtained from the cross-correlation of both signals, which clearly shows how the white light scattering layer becomes significantly more permeable to CO₂ above 40° C. The dashed line corresponds to the fit of a second-order polynomial and is shown as a guide to illustrate the trend.

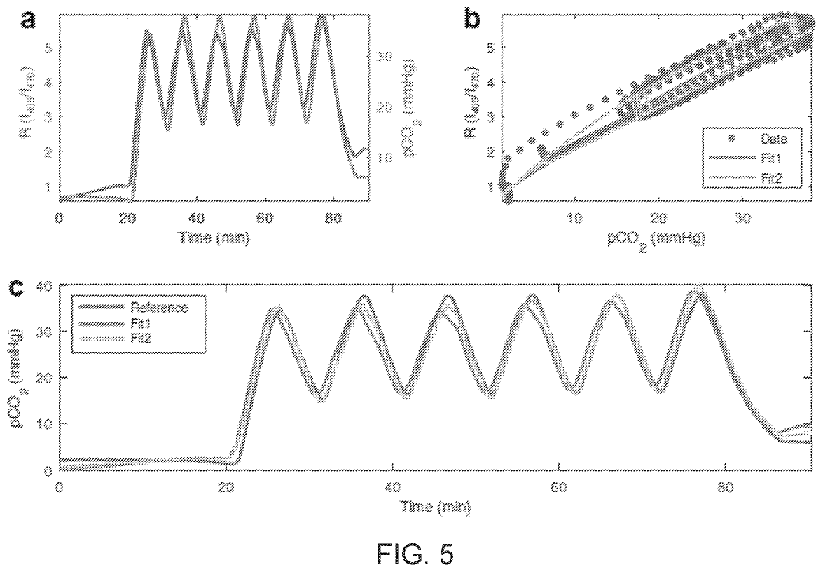

In order to calibrate the prototype, as well as test for cyclability, we programmed a multiple, 5 minute-long, cycle sequence into the gas mixer. The measurement shown in FIG. 5, panel a, was carried out at an average temperature of 45° C. The fluorescence ratio showed a sensitive and fast response to changes in CO₂ during the 90 minute measurement.

Following the work in [Ref. 25], the dependence of pCO₂ on the fluorescence ratio R obeys the following equation:

$${pCO}_{2} = \frac{A \cdot R - B}{- C \cdot R + D} \qquad ( 4 )$$

where the constants (A, B, C, D)>0 are obtained by combining different kinetic rates and material parameters such as quantum yield, etc. (see [Ref. 25]). We fit Equation (4) to the data in the figure using a script written for GNU Octave [Ref. 31]. As can be seen in FIG. 5, panel b, the model captures the trend well (R²=0.9518) and produces a pCO₂ estimate (Fit1 in FIG. 5, panel c), which quantitatively follows the reference sensor's readings. Although this fit captures the average trend of the response, the estimated pCO₂ is always either over-or under-estimated due to the different rates for protonation/de-protonation, as mentioned above.

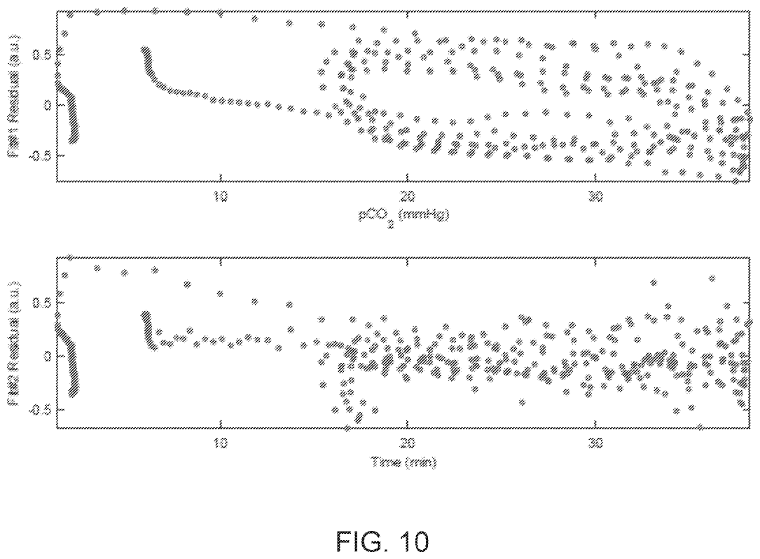

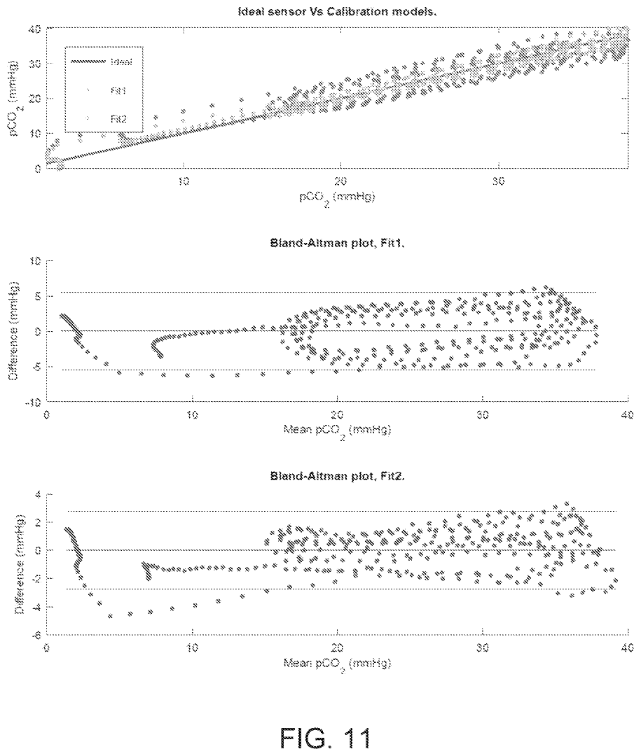

Given these data, we can propose a second model (Fit2 in FIG. 5, panel b), which also uses Equation (4), but with different calibration parameters (A, B, C, D) depending on whether the derivative of the fluorescence ratio is positive (CO₂ increasing) or negative (CO₂ decreasing). This model improves on the over-/under-estimation of Fit1 (higher R²=0.9808) and follows closely the reference pCO₂ readings, as can be seen in FIG. 5, panel c, and the residual plot in FIG. 10. The standard deviation o of the difference between the reference sensor and our pCO₂ estimate supports what is seen in FIG. 5, panel c, with σ_{Fit1}=5.50 mmHg and σ_{Fit2}=2.73 mmHg (see FIG. 11 for Bland-Altman plots of the models). Further, the results from Fit1 were found to lag behind the reference reading by 37 seconds, while Fit2 showed no lag.

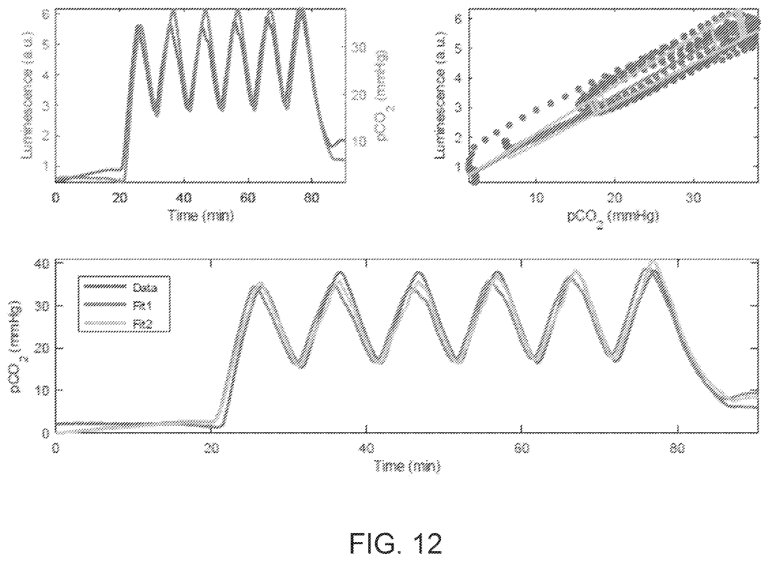

The work in [Ref. 25] proposed a linear dependence with CO₂, with the fitting coefficients corresponding to the ratios of the different kinetic rates. A linear dependence does not capture the curvature of our data (see FIG. 12), most likely due to the added complexity of CO₂ diffusing through the different layers of the film.

The estimated sensitivity of our device considering the simpler linear relationship between R and pCO₂ yields a rate of change for R of:

$$\frac{Δ R}{Δ {pCO}_{2}} = \frac{( {R}^{max} - {R}^{min} )}{( {pCO}_{2}^{max} - {pCO}_{2}^{min} )} = 0.13 / mmHg \qquad ( 5 )$$

or a percentage rate of change:

$$\frac{Δ R \%}{Δ {pCO}_{2}} = 100 \cdot \frac{( {R}^{max} / {R}^{min} - 1 )}{( {pCO}_{2}^{max} - {pCO}_{2}^{min} )} = 14.2 \% / mmHg \qquad ( 6 )$$

obtained using the data from FIG. 5.

In summary, our ratiometric readings and calibration algorithm are able to reliably obtain pCO₂ readings during multiple hours of measurements without being affected by photobleaching, and do so without deviating from the bench-top, commercial pCO₂ sensors. Depending on the application, longer monitoring times may be required, and the use of the films could be limited by HPTS photobleaching. This could be easily solved by periodically replacing films or reducing the sampling frequency of the device so the films are probed less frequently and, consequently, yield high signals for longer wear times.

In this Example, we aimed to explore different HPTS-based ion pairs within different support matrices in order to identify optimal materials to be used with optical wearable devices for real-time monitoring of transcutaneous CO₂ partial pressure. A thorough characterization of our materials, and most notably (HPTS)/(TOA)₄ embedded within a PPMA matrix, showed them to be highly sensitive in the physiological CO₂ range (0-50 mmHg). These films were found to be intrinsically insensitive to changes in humidity, a consideration that is important for skin-worn devices, as well as photostable during continuous sampling for long periods. We incorporated these materials into a medical-grade, multi-layer film to measure CO₂ transcutaneously, carried out with an optical wearable device prototype. We proposed a detection and calibration methodology, relying on the formulation's isosbestic point within the physiological CO₂ range, which can robustly and reversibly detect CO₂ changes within the physiological range, while compensating for fluorescence intensity changes due to photobleaching, as well as motion artifacts. The response speed of our films is comparable to commercial NDIR CO₂ sensors and is ideal to detect physiological changes, which occur on a timescale of minutes. Our Example shows great potential to develop commercial, miniaturized wearable devices to monitor tissue CO₂ partial pressure at the skin surface. Further, both the device and the film structure, based on our team's approach to measure transcutaneous oxygenation, have already been validated clinically, detecting changes in tissue oxygenation non-invasively in different physiological scenarios. Our prior work, along with the results shown here present an ideal platform to obtain dual O₂/CO₂ wearable sensors.

Based on this Example, it is contemplated that one can aim to develop a new light scattering layer that is highly permeable to CO₂ [Ref. 32] in a wide temperature range so as not to require heating for transcutaneous applications. Alternatively, our devices could incorporate a small heating element to achieve temperatures around 42-45° C., which are typically used in standard-of-care transcutaneous gas sensing devices. Based on this Example, it is contemplated that one can aim to adapt our technology to create wearable or portable devices of different form factors, allowing us to continuously monitor both O₂ and CO₂ on the skin surface [Ref. 26,33], in exhaled breath (capnography), and within arterial lines or muscle tissue [Ref. 27].

Non-limiting beneficial features demonstrated in this Example include:
a formulation of an (HPTS)/(TOA)₄ ion pair in a PPMA matrix, which is mechanically robust and hydrophobic, therefore not affected by humidity;
a wearable device that is a lightweight, standalone device, comprising electronics such as a microcontroller, LEDs, PIN photodiode, thermistor, amplifiers, ADC chips and passive components (R, L, C);
the use of multiple LEDs with different excitation wavelengths allows for a normalized or ratiometric luminescence which provides robustness against motion artifacts, ambient light changes, photobleaching, etc., compared to simple intensity or colorimetric based measurements, which is relevant for portable health applications;
hardware using: low power and voltage components: microcontroller vs. PC, LEDs vs. Xe lamp, PIN diode vs. CCD detector (DSLR Camera), live readings vs. post processing required, and continuous point measurement vs. image;
hardware which is a wearable, standalone device which can obtain CO₂ readings and communicate them to computers or smartphones wirelessly in contrast to prior devices require a computer to obtain the readings;
the disposable, single-use CO₂ sensing films require only a small volume (no dead-space) of air to sample CO₂, and hence allow for fast equilibration and readings, which also allows for a commercial product based on our Example to be more compact in contrast to NDIR CO₂ sensors which require large optical paths and therefore large volumes of air for enough infrared light to be absorbed at low concentrations of CO₂;
the formulation is humidity insensitive which is advantageous in that water vapor in air also affects the readings of infrared based sensors; and.
the measurement approach can also be readily combined with optical oxygen sensors for dual readout of both CO₂ and O₂.

### REFERENCES

1. Patel, S.; Miao, J. H.; Yetiskul, E.; Anokhin, A.; Majmundar, S. H., Physiology, Carbon Dioxide Retention. In StatPearls; StatPearls Publishing LLC.: Treasure Island, FL, USA, 2022.
2. Casati, A.; Squicciarini, G.; Malagutti, G.; Baciarello, M.; Putzu, M.; Fanelli, A., Transcutaneous monitoring of partial pressure of carbon dioxide in the elderly patient: A prospective, clinical comparison with end-tidal monitoring. J. Clin. Anesth. 2006, 18, 436-440.
3. Umeda, A.; Ishizaka, M.; Ikeda, A.; Miyagawa, K.; Mochida, A.; Takeda, H.; Takeda, K.; Fukushi, I.; Okada, Y.; Gozal, D., Recent Insights into the Measurement of Carbon Dioxide Concentrations for Clinical Practice in Respiratory Medicine. Sensors 2021, 21, 5636.
4. Huttmann, S. E.; Windisch, W.; Storre, J. H., Techniques for the measurement and monitoring of carbon dioxide in the blood. Ann. Am. Thorac. Soc. 2014, 11, 645-652.
5. Tipparaju, V. V.; Mora, S. J.; Yu, J.; Tsow, F.; Xian, X., Wearable Transcutaneous CO₂ Monitor Based on Miniaturized Nondispersive Infrared Sensor. IEEE Sens. J. 2021, 21, 17327-17334.
6. Berkenbosch, J. W.; Lam, J.; Burd, R. S.; Tobias, J. D., Noninvasive monitoring of carbon dioxide during mechanical ventilation in older children: End-tidal versus transcutaneous techniques. Anesth. Analg. 2001, 92, 1427-1431.
7. Casati, A.; Salvo, I.; Torri, G.; Calderini, E., Arterial to end-tidal carbon dioxide gradient and physiological dead space monitoring during general anaesthesia: Effects of patients' position. Minerva Anestesiol. 1997, 63, 177-182.
8. Wyatt, J.; Edwards, A.; Cope, M.; Delpy, D.; McCormick, D.; Potter, A.; Reynolds, E., Response of cerebral blood volume to changes in arterial carbon dioxide tension in preterm and term infants. Pediatr. Res. 1991, 29, 553-557.
9. Senn, O.; Clarenbach, C. F.; Kaplan, V.; Maggiorini, M.; Bloch, K. E., Monitoring carbon dioxide tension and arterial oxygen saturation by a single earlobe sensor in patients with critical illness or sleep apnea. Chest 2005, 128, 1291-1296.
10. Bendjelid, K.; Schütz, N.; Stotz, M.; Gerard, I.; Suter, P. M.; Romand, J. A., Transcutaneous pCO₂ monitoring in critically ill adults: Clinical evaluation of a new sensor. Crit. Care Med. 2005, 33, 2203-2206.
11. Nishiyama, T.; Nakamura, S.; Yamashita, K., Comparison of the transcutaneous oxygen and carbon dioxide tension in different electrode locations during general anaesthesia. Eur. J. Anaesthesiol. 2006, 23, 1049-1054.
12. Palmisano, B. W.; Severinghaus, J. W., Transcutaneous pCO₂ and pO₂: A multicenter study of accuracy. J. Clin. Monit. 1990, 6, 189-195.
13. Finžgar, M.; Frangež, H. B.; Cankar, K.; Frangež, I., Transcutaneous application of the gaseous CO₂ for improvement of the microvascular function in patients with diabetic foot ulcers. Microvasc. Res. 2021, 133, 104100.
14. Dervieux, E.; Theron, M.; Uhring, W. Carbon Dioxide Sensing-Biomedical Applications to Human Subjects. Sensors 2021, 22, 188.
15. Escobedo, P.; Fernández-Ramos, M.; López-Ruiz, N.; Moyano-Rodríguez, O.; Martínez-Olmos, A.; Pérez de Vargas-Sansalvador, I.; Carvajal, M.; Capitán-Vallvey, L.; Palma, A., Smart facemask for wireless CO₂ monitoring. Nat. Commun. 2022, 13, 1-12.
16. Malins, C.; MacCraith, B. D., Dye-doped organically modified silica glass for fluorescence based carbon dioxide gas detection. Analyst 1998, 123, 2373-2376.
17. Wolfbeis, O. S.; Kovács, B.; Goswami, K.; Klainer, S. M., Fiber-optic fluorescence carbon dioxide sensor for environmental monitoring. Microchim. Acta 1998, 129, 181-188.
18. Mills, A.; Chang, Q., Fluorescence plastic thin-film sensor for carbon dioxide. Analyst 1993, 118, 839-843.
19. Mills, A.; Yusufu, D., Highly CO₂ sensitive extruded fluorescent plastic indicator film based on HPTS. Analyst 2016, 141, 999-1008.
20. Chu, C. S.; Syu, J. J., Optical sensor for dual sensing of oxygen and carbon dioxide based on sensing films coated on filter paper. Appl. Opt. 2017, 56, 1225-1231.
21. Roussakis, E.; Cascales, J. P.; Marks, H. L.; Li, X.; Grinstaff, M.; Evans, C. L., Humidity-Insensitive Tissue Oxygen Tension Sensing for Wearable Devices. Photochem. Photobiol. 2020, 96, 373-379.
22. Li, X.; Roussakis, E.; Cascales, J. P.; Marks, H. L.; Witthauer, L.; Evers, M.; Manstein, D.; Evans, C. L., Optimization of bright, highly flexible, and humidity insensitive porphyrin-based oxygen-sensing materials. J. Mater. Chem. C 2021, 9, 7555-7567.
23. Wang, X.d.; Wolfbeis, O. S., Optical methods for sensing and imaging oxygen: Materials, spectroscopies and applications. Chem. Soc. Rev. 2014, 43, 3666-3761.
24. Burke, C. S.; Markey, A.; Nooney, R. I.; Byrne, P.; McDonagh, C., Development of an optical sensor probe for the detection of dissolved carbon dioxide. Sens. Actuators B Chem. 2006, 119, 288-294.
25. Zhu, Q.; Aller, R. C.; Fan, Y., A new ratiometric, planar fluorosensor for measuring high resolution, two-dimensional pCO₂ distributions in marine sediments. Mar. Chem. 2006, 101, 40-53.
26. Cascales, J. P.; Roussakis, E.; Witthauer, L.; Goss, A.; Li, X.; Chen, Y.; Marks, H. L.; Evans, C. L., Wearable device for remote monitoring of transcutaneous tissue oxygenation. Biomed. Opt. Express 2020, 11, 6989-7002.
27. Witthauer, L.; Cascales, J. P.; Roussakis, E.; Li, X.; Goss, A.; Chen, Y.; Evans, C. L., Portable oxygen-sensing device for the improved assessment of compartment syndrome and other hypoxia-related conditions. ACS Sens. 2020, 6, 43-53.
28. Van Rossum, G.; Drake, F. L., Jr., Python ReferenceManual; Centrum voorWiskunde en Informatica: Amsterdam, The Netherlands, 1995.
29. Pinnau, I.; Morisato, A.; He, Z., Influence of Side-Chain Length on the Gas Permeation Properties of Poly (2-alkylacetylenes). Macromolecules 2004, 37, 2823-2828.
30. Chu, C. S.; Lo, Y. L., Highly sensitive and linear optical fiber carbon dioxide sensor based on sol-gel matrix doped with silica particles and HPTS. Sens. Actuators B Chem. 2009, 143, 205-210.
31. Eaton, J. W.; Bateman, D.; Hauberg, S.; Wehbring, R., GNU Octave Version 3.8.1 Manual: A High-Level Interactive Language for Numerical Computations; CreateSpace Independent Publishing Platform: Scotts Valley, CA, USA, 2014; ISBN 1441413006.

32. Préfol, T.; Gain, O.; Sudre, G.; Gouanvé, F.; Espuche, E., Development of Breathable Pebax®/PEG Films for Optimization of the Shelf-Life of Fresh Agri-Food Products. Membranes 2021, 11, 692.

33. Marks, H.; Bucknor, A.; Roussakis, E.; Nowell, N.; Kamali, P.; Cascales, J. P.; Kazei, D.; Lin, S. J.; Evans, C. L., A paintable phosphorescent bandage for postoperative tissue oxygen assessment in DIEP flap reconstruction. Sci. Adv. 2020, 6, eabd1061.

The citation of any document or reference is not to be construed as an admission that it is prior art with respect to the present invention.

Thus, the present invention provides devices, materials, and methods for analyte monitoring, and more particularly to devices, materials, and methods for carbon dioxide monitoring.

In light of the principles and example embodiments described and illustrated herein, it will be recognized that the example embodiments can be modified in arrangement and detail without departing from such principles. Also, the foregoing discussion has focused on particular embodiments, but other configurations are also contemplated. In particular, even though expressions such as “in one embodiment”, “in another embodiment”, “in other embodiments”, “in some embodiments”, or the like are used herein, these phrases are meant to generally reference embodiment possibilities, and are not intended to limit the invention to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another. Although the invention has been described in considerable detail with

reference to certain embodiments, one skilled in the art will appreciate that the present invention can be used in alternative embodiments to those described, which have been presented for purposes of illustration and not of limitation. Therefore, the scope of the appended claims should not be limited to the description of the embodiments contained herein.

1. A device for carbon dioxide monitoring, the device comprising:
a photoluminescent carbon dioxide-sensitive probe comprising a polymer matrix and a sensing dye;
a photon source configured to direct photons at the probe;
a photodetector configured to detect light emitted from the probe when the photon source directs photons at the probe; and
a controller in electrical communication with the photon source and the photodetector, the controller being configured to execute a program stored in the controller to calculate a level of carbon dioxide adjacent the probe from an electrical signal received from the photodetector,
wherein the polymer matrix comprises a polymer selected from the group consisting of acrylate polymers, methacrylate polymers, polyurethane polymers, and blends and copolymers thereof.

2. The device of claim 1 wherein:
the polymer matrix comprises a hydrophobic polymer.

3. The device of claim 1 wherein:
the polymer matrix comprises a polymer selected from the group consisting of alkyl methacrylate polymers.

4. (canceled)

5. A device for carbon dioxide monitoring, the device comprising:
a photoluminescent carbon dioxide-sensitive probe comprising a polymer matrix and a sensing dye;
a photon source configured to direct photons at the probe;
a photodetector configured to detect light emitted from the probe when the photon source directs photons at the probe;
a carbon dioxide permeable light redirection layer, wherein the carbon dioxide-sensitive probe is positioned between the light redirection layer and the photodetector; and
a controller in electrical communication with the photon source and the photodetector, the controller being configured to execute a program stored in the controller to calculate a level of carbon dioxide adjacent the probe from an electrical signal received from the photodetector.

6. The device of claim 5 wherein:
the light redirection layer can scatter light.

7. The device of claim 5 wherein:
the light redirection layer can reflect light.

8. The device of claim 5 wherein:
the light redirection layer comprises a silicone film including a pigment.

9. (canceled)

10. (canceled)

11. (canceled)

12. (canceled)

13. The device of claim 5 further comprising:
a partially air-impermeable layer positioned between the carbon dioxide-sensitive probe and the photodetector.

14. The device of claim 5 further comprising:
a partially air-impermeable outer layer.

15. (canceled)

16. (canceled)

17. A device for carbon dioxide monitoring, the device comprising:
a photoluminescent carbon dioxide-sensitive probe comprising a polymer matrix and a sensing dye;
a first photon source configured to direct photons at a first wavelength at the probe;
a second photon source configured to direct photons at a second wavelength at the probe, wherein the second wavelength is different from the first wavelength;
a photodetector configured to detect light emitted from the probe when the first photon source and the second photon source direct photons at the probe; and
a controller in electrical communication with the first photon source and the second photon source and the photodetector, the controller being configured to execute a program stored in the controller to calculate a level of carbon dioxide adjacent the probe from an electrical signal received from the photodetector.

18. The device of claim 17 wherein:
an excitation spectrum of the sensing dye has an isosbestic point at the first wavelength.

19. The device of claim 17 wherein:
directing photons at the first wavelength at the probe provides a normalization factor to account for variations in brightness of the polymer matrix.

20. The device of claim 19 wherein:
the controller is configured to execute the program stored in the controller to calculate the level of carbon dioxide adjacent the probe using a fluorescence ratio providing a metric that is proportional to the level of carbon dioxide adjacent the probe and is normalized using the normalization factor.

21. (canceled)

22. (canceled)

23. The device of claim 17 wherein:
the controller is configured to execute the program stored in the controller to calculate the level of carbon dioxide adjacent the probe by detecting intensity of the emission excited at the first wavelength and the second wavelength.

24. The device of claim 17 wherein:
the first photon source and the second photon source are modulated by a sinusoidal voltage and an intensity of a fluorescent response of the sensing dye is defined as an amplitude of a measured sinusoidal response, which is extracted via multiple linear regression.

25. The device of claim 1 wherein:
the photodetector detects a fluorescence response of the sensing dye that provides tissue pCO₂.

26. (canceled)

27. (canceled)

28. (canceled)

29. The device of claim 1 wherein:
the carbon dioxide-sensitive probe comprises a phase transfer reagent co-embedded with the sensing dye within the polymer matrix.

30. (canceled)

31. (canceled)

32. (canceled)

33. (canceled)

34. (canceled)

35. (canceled)

36. (canceled)

37. The device of claim 1 wherein:
the carbon dioxide-sensitive probe has a storage stability such that no significant decrease of sensitivity when the carbon dioxide-sensitive probe is stored under ambient and dark conditions for at least seven days.

38. The device of claim 1 wherein:
the device is an optical transcutaneous device.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51 (canceled)

52. (canceled)

53. (canceled)

54. (canceled)

55. A method for detecting a concentration of an analyte, the method comprising:
(a) providing a device comprising: (i) a probe including a polymer matrix and a sensing dye, (ii) a first photon source configured to direct photons at a first wavelength at the probe, (iii) a second photon source configured to direct photons at a second wavelength at the probe, wherein the second wavelength is different from the first wavelength, and (iv) a photodetector configured to detect light emitted from the probe when the first photon source and the second photon source direct photons at the probe; and
(b) calculating a concentration of the analyte adjacent the probe based on the light emitted from the probe detected by the photodetector and an isosbestic point from an excitation spectrum of the sensing dye at the first wavelength.

56. (canceled)

57. (canceled)

58. (canceled)

59. (canceled)

60. (canceled)

61. (canceled)

62. (canceled)

63. (canceled)

64. (canceled)

65. (canceled)
